(19) United States
(12) Patent Application Publication
(10) Pub. No.: US 2024/0277975 A1
(43) Pub. Date: Aug. 22, 2024

(54) MEDICAL DEVICE WHICH INCLUDES A BALLOON MODULE

(71) Applicant: HOOP MEDICAL LIMITED, Ardcavan (IE)

(72) Inventor: Edward Charles MUDGE, London (GB)

(73) Assignee: HOOP MEDICAL LIMITED, Ardcavan (IE)

(21) Appl. No.: 18/571,011

(22) PCT Filed: Jun. 16, 2022
(86) PCT No.: PCT/IB2022/055595

§ 371 Date: Dec. 15, 2023

(30) Foreign Application Priority Data
Jun. 18, 2021 (ZA) 2021/04170

(51) Int. Cl. A61M 25/10

(52) U.S. Cl. CPC A61M 25/1002; A61M 25/1011; A61M 2025/1015; A61M 2025/105; A61M 2025/1059; A61M 2025/1061; A61M 2025/1065; A61M 2025/1086; A61M 2205/7545

(57) ABSTRACT

The invention provides a medical device (10) for use within the human body, which includes a catheter tube (12) having a distal end and a proximal end, an inflation passage (30) defined within the catheter tube having an outlet (32), at least one balloon module (38) which has a first end (40) and a second end (42) which engage the catheter tube to envelop the outlet, and between which an outer wall (50), a closed perimeter (44), and an inner wall (52) is disposed to define an inflation enclosure (55), and a cavity (53) defined within the inner wall, wherein the balloon component is inflatable by inflow of an inflation fluid through the inlet into the inflation enclosure from a deflated state to an inflated state, wherein the balloon has at least one bond zone (54) within which the inner wall is bonded directly to the outer wall, and wherein, in the inflated state, the outer wall and at least part of the inner wall move radially outwardly relatively to the longitudinal axis of the catheter to cause expansion of the cavity and to radially space apart the closed perimeter from the longitudinal axis of the catheter.

### FIELD OF THE INVENTION

The invention relates to a medical device for use within the human body, such as, for example, a balloon catheter, an introducer sheath or an implant, which includes a hollow balloon component or module which can be actuated between an inflated state and un-inflated or contracted state by means of an inflation lumen, and which provides a configuration that can be used for many medical applications.

### BACKGROUND TO THE INVENTION

Medical balloon components are included in many different types of medical devices, to treat a broad variety of medical conditions and these balloon components take on a range of shapes and forms.

Hereinafter, the term “component” and “module” are used interchangeably to refer to a discretely inflatable balloon unit.

A typical balloon catheter includes a balloon component which when inflated expands to do useful work within the human body via percutaneous access. The balloon catheter includes a balloon component which can be manipulated or actuated by means of fluid injection, between an un-inflated or contracted state in which state it has a relatively small diameter (such that it may be easily inserted into the human body through an introducer sheath within the groin for example) to and an inflated or deployed state, in which it has a larger diameter. The balloon component performs work in reaching a fully inflated or deployed state. For example the balloon component can exert a radially outward force onto the inner portion of a compressed/crimped metal stent or implant in order to dilate said implant into its deployed state. Examples of these more typical balloon catheters include PTA (Percutaneous Transluminal Angioplasty) balloon catheters, Foley balloon catheters and TAVI (Transcatheter Aortic Valve Implantation) balloon catheters.

Balloon components are sometimes subjected to a Pleat and Wrap process whereby longitudinal pleats or folds are induced to the balloon component and it is then wrapped around the catheter shaft, to minimise the crossing profile of the balloon in the un-inflated or contracted state.

Balloon components are also sometimes used on introducer sheaths/ports like for example the Kii balloon blunt-tip access system by Applied Medical which makes use of a balloon component to provide anchorage with the human body during laparoscopic procedures.

On many devices including a balloon component or balloon component assembly, the balloon component is mounted near to or at the distal end of the catheter shaft, However, the balloon component can also be mounted at any mid-way point along the shaft as is the case with the Kii Access System by Applied Medical. The catheter shaft usually runs some length (usually between 10 cm and 120 cm) and on or near the proximal end of the catheter shaft is an inflation port. This inflation port is in fluid communication with the balloon component. The catheter shaft often includes a guidewire lumen and/or a working channel as is appreciated by those familiar with devices within the minimally-invasive medical devices industry.

The balloon component is inserted into the human body in an un-inflated state. With the smaller diameter, a smaller incisions or access route can be made with obvious medical advantages. The balloon component is connected to a means of inflation (such as a syringe or an auto-inflator) via an inflation lumen often running the entire length of the catheter. When the balloon component has been advanced into position, the means of inflation is actuated and the pressure within the inflation port, the inflation lumen and the balloon component increases. This pressure increase is used to do work. The work, with respect to a “compliant balloon” component, is to occlude a vessel or orifice (e.g. Foley catheters), and, with respect to a “non-compliant”, is to deploy a stent or to dilate a stenosed artery. An example for this application is a PTA catheter.

Many balloon catheters remain in the body for only a short time. PTA, PTCA (Percutaneous Transluminal Coronary Angioplasty) and TAVI balloon catheters are examples that typically remain in the body for a matter of minutes. Other balloon catheters, like the Foley catheter or the Intra-Aortic Balloon Pump (IABP) catheter, can remain in the human body for a number of weeks or even months.

Balloon catheter designers and engineers often refer to “compliant”, “semi-compliant” and “non-compliant” balloon components. These three groups are intended to describe the way in which the balloon component changes in diameter as it is inflated to greater and greater pressures or as it is inflated with greater and greater volumes of inflation fluid (for example saline or air).

Compliant balloon components increase significantly in diameter as they are inflated. Compliant balloon components are generally made from relatively soft and elastic polymer or rubber materials with shore durometer hardness in the 20 A to 90 A range, such as silicones, latex, thermoplastic polyurethanes (TPUs) or thermoplastic elastomers (TPEs). These elastic materials can elongate or strain significantly as more inflation fluid is added within the balloon component. Balloon components made from these relatively soft materials typically inflate to take on a largely rounded, spherical or long-spherical shape when inflated to pressures greater than around 3 psi to 20 psi. Compliant balloons typically inflate at fairly low pressures in the 3 psi to 20 psi range. Indeed, some of the materials used for these types of balloon components have ultimate elongation values of around 500% (like TPUs) to around 1500% (like TPEs and Latex). Hence a compliant balloon can grow in diameter from say 3 mm in the un-inflated state to say 20 mm in the inflated state without rupture and at a relatively low inflation pressure. This feature can be useful for applications requiring occlusion or mechanical anchorage from the balloon component. It can also be useful for applications that require the balloon component to make contact with the target anatomy or device while exerting a relatively low force on that target anatomy or device to be deployed like for example a urinary balloon catheter like the Foley catheter.

Non-compliant balloon components on the other hand do not increase significantly in diameter as they are inflated to greater and greater pressures. The strong and relatively stiff materials from which these balloon components are made do not elongate or strain significantly as more inflation fluid is added within the balloon component. Consequently, the pressure within the balloon component increases significantly as more fluid is added, while the diameter of the balloon does not increase significantly. This can be useful for applications such as dilation of the target anatomy or device where a relatively high force (and high pressure) is required and a specific target diameter must be achieved, like for example in the deployment of a balloon expandable TAVI stented heart valve. Non-compliant balloon components are often made from relatively hard and in-elastic and strong (high ultimate tensile strength) polymer materials with shore durometer hardness in the 70 D to 90 D range such as, for example, Polyethylene Terephthalate (PET) and Nylon 12 (PA12). Balloon components made from these relatively hard materials typically hold their shape and diameter when inflated to relatively high pressures in the 4 atm to 40 atm range.

Semi-compliant balloon components are those which fall into a group that is in between the compliant and non-compliant groups. Semi-compliant balloon components grow more in diameter that non-compliant balloon components when inflated to higher pressures. Semi-compliant balloons typically hold pressures that are in the range of 1 atm to 20 atm. They can sometimes be configured to provide growth at relatively high pressures can thus be used to provide a “one size fits all” type device, where the clinician can choose (usually within a fairly narrow range) the diameter to which they want to inflate the balloon, usually using a diameter vs pressure chart or table provided by the device manufacturer within the instructions for use.

A standard shaped balloon component (like those used in PTA or PTCA procedures for example) can be described as having geometric features including a first or distal neck (a largely tubular or hollow cylindrical portion also known as a “leg” or “tail”) which transitions into a first or proximal conical portion (a conical or hemi-spherical region with a smaller diameter at the neck-to-cone transition and a larger diameter at the cone-to-mid-portion transition) which tapers wider to transition into a mid-portion or “working-length” of the balloon (which typically has an outer surface which is largely cylindrical in shape, and is region of the balloon component, when inflated, with the largest diameter). Distally, the mid-portion transitions to a second or distal conical portion which tapers narrower (or tapers down) to a second or distal neck which is largely tubular like the first neck.

The first and second necks are the portions of the balloon component that typically bond to the catheter shaft or shafts.

A conical balloon component has a shape similar to the standard balloon shape described above, but with no mid-portion or “working length”.

A spherical balloon component can be described as having two hemi-spherical shaped cone sections connected to one another. So, for conical and spherical balloons, the conical portion can be described as the working length of the balloon since it is the only portion of the balloon that is not bonded or fixed to the catheter shaft.

A long spherical balloon component is like a spherical balloon component, but with a cylindrical working length or mid-portion positioned between the hemi-spherical cones. In other words a long spherical balloon component is like a standard shape balloon component but with rounded cones or hemi-spherical cones.

For example, a compliant balloon component can change shape significantly between the un-inflated state and the inflated state. For example, a compliant balloon can have a standard shape when partially inflated (to some low pressure like 0.1 psi to 0.5 psi) which then grows and changes into a spherical shape when fully inflated.

Dilation balloon components are often used to expand or open up targets such as blocked or stenosed vessels or crimped-down metal stented implants for example. In some instances the force needed to dilate the target is substantial and a high-pressure tolerant non-compliant balloon component is required.

Dilation balloons are often made from relatively hard and in-elastic and strong (high ultimate tensile strength) polymer materials like those described as being “non-compliant” or “semi compliant” above. Balloons made from these relatively hard materials typically hold their shape when inflated to relatively high pressures in the 6 atm to 30 atm range, and because they can withstand these high pressure they are better suited to dilation applications which typically require moderate to high outward radial force from the balloon component.

Occlusion balloons are used to block or occlude vessels or orifices within the human body. Occlusion balloons often have an overall spherical or long-spherical shape. The “cones” in these types of balloons are sometimes formed to have a conical shape when pressurised at a very low pressure like 0.3 psi for instance. But due to the soft and compliant nature of the materials used for occlusion balloons, they typically lose their shape and “round out” when inflated to higher pressures (like 5 psi to 20 psi for example). A long-spherical balloon has a shape similar to standard balloon but with rounded hemi-spherical “cones” on both ends of the working length. A spherical balloon typically has no cylindrical mid-portion, but rather each cone transitions into the other directly.

Occlusion balloons are often made from relatively soft and elastic polymer or rubber materials as detailed in the compliant balloons description above. Balloons made from these relatively soft materials typically grow to take on a largely round or spherical or long-spherical shape when inflated to pressures greater than around 5 psi to 20 psi. Said differently, an occlusion balloon does not typically hold its shape definition throughout inflation, but rather expands towards a rounder version of its original shape.

There are also other more exotic balloon catheters fitted with balloon components with somewhat unusual shapes or configurations. For example, valvuloplasty balloon components (used to dilate or radially-expand heart valves) sometimes have an hourglass shape (where the working portion of the balloon includes a central “waist” region with a smaller diameter) that allows the balloon to fit more snugly within the target anatomy (a heart valve in this example)

Cryoablation balloon catheters are sometimes fitted with 2 balloon components, the inner balloon positioned radially inward from the outer balloon. In other words, the inner balloon is positioned within the outer balloon. The Arctic Front system is an example of a balloon catheter including two balloons as described here.

Some devices include balloon components which have an inverted or everted region. The Cook Cervical Ripening Balloon is an example of this. By inverting one end (for example the distal neck) of a balloon component, a favourable shape balloon assembly or balloon module can be achieved, and functionality of the medical device can be improved.

In practice the proximal neck of a standard shaped balloon component is sometimes larger in diameter than the distal neck in order to allow bonding of the proximal balloon neck to an outer catheter shaft and bonding of the distal balloon neck to a smaller diameter inner catheter shaft. The annular space between the inner and outer shafts is often used as the inflation lumen to inflate the balloon component.

Those familiar with the art of balloon catheter design and manufacture will know that in other instances, the balloon component on a balloon catheter may have proximal and distal necks with the same or similar diameters and may be mounted on a multi-lumen catheter shaft which can accommodate the proximal and distal necks having similar diameters. One of the lumens in the multi-lumen shaft is used as an inflation lumen for the balloon component. The inflation lumen is exposed often by means of one or more “skives” or cuts or holes at a position that lies between the proximal and distal necks of the balloon component such that the inner portion of the balloon component is in fluid communication with the inflation lumen which is in fluid communication with the inflation port.

Potential use cases for a conical or spherically shaped balloon component, providing a hollow funnel, include those discussed in the prior art namely for occlusion of vessels and orifices within the human body and or for retrieval of devices or waste matter or emboli from within the human body.

As known in the prior art, inverting or everting one end of a standard shaped balloon component (including one neck and one cone and perhaps a portion of the working length) a hollow balloon component with a shape similar to a funnel can be realised. In practice, this prior art is at best, limited and at worst, fundamentally flawed.

In practice (as is known to those who have prototyped compliant inverted or everted balloons), a compliant or thin-walled occlusion balloon such as that described above and will collapse inwardly about a distal end.

The prior art includes a funnel shaped balloon with at least one inverted/everted neck region as described in CA2492020A1 (Gore). This document describes a method and apparatus for removing emboli during an angioplasty, stenting or surgical procedure. The apparatus comprises a catheter having a funnel-shaped occlusion balloon of uniform thickness disposed on a distal end of the catheter. The occlusion balloon is fused to the distal end so that it provides a substantially seamless flow transition into a working lumen of the catheter. Additionally, a distal edge of the occlusion balloon is configured to be in close proximity with an inner wall of a vessel to facilitate blood flow into the catheter and efficiently remove emboli.

The problem with the apparatus of Gore is that, when inflated the balloon will have a rounded out distal taper. The balloon is unlikely to have a conical shape when inflated because it is a compliant (or thin walled) balloon. This inevitable “rounding out” represents a departure from the intended design, which is a conical distal taper to allow a funnel shape into which devices or emboli may more easily be retracted.

There are some less common balloon catheters which make use of a plurality of balloon components assembled together in parallel such that the device allows perfusion through a vessel while the balloons are inflated there-in. Examples of these include the Bard True Flow balloon catheter which is used to pre-dilate the aortic valve prior to implantation of a TAVI valve; the Gore Tri-Lobe balloon catheter which is used to “re-model” or “post-dilate” a self-expanding stent graft within the thoracic abdominal aorta; and, more recently, the Disa Medinotec Trachealator Airway Dilation Balloon which is used to dilate the trachea. All of these devices are designed such that when inflated the balloon module or balloon assembly expands radially outward to dilate the target anatomy or target device by applying an outward radial force from within (much like a standard dilation type balloon) but whilst still allowing fluid or blood to flow through an at least partially hollow section of the inflated balloon module or balloon assembly, thus permitting perfusion during use.

Having a device which comprises multiple balloon components assembled in parallel has two key drawbacks. Since the structure to be dilated by the balloon component, either an associated device to be deployed or a part of a patient's anatomy, is often substantially cylindrical in shape, a device comprising multiple parallel balloons will make contact with that cylinder along a few discrete lines or contact-regions, which can be undesirable. For example if three balloons are positioned in parallel (as is the case with the Gore Tri-Lobe balloon catheter) the target implant may be forced into a rounded-triangle shape (when viewed in cross section) rather than a more circular shape as is desired. Moreover, the individual balloon components which make up the multi-balloon module are typically fairly high value parts. On a standard PTA balloon catheter device for example, the balloon component can cost more than the other parts combined (including catheter shafts, inflation lures/ports). So a design that relies on using multiple components will typically have a significantly higher cost of parts which can be commercially undesirable.

Other prior art shows helical balloons which are wound into a round-tubular shape to form a hollow perfusion balloon module. These hollow helical perfusion balloons are shown to have applications including balloon dilation applications like valvuloplasty, stent deployment and balloon expandable TAVI valve deployment, tasks which are possible while maintaining perfusion thanks to the hollow central portion within the windings of the helix. One drawback of some of these helical balloons is that they typically enquire a separate frame or connector to create structural linkages between each winding of each helical coil to prevent the coil from crumpling or collapsing when exposed to a significant resistive force (such as an un-deployed metal stent for example). These additional components add material and bulk to the balloon module assembly. Moreover, any additional structural component such as a frame may itself require a certain dilation force from the helical balloon in order to expand said frame, which represents an inefficiency or a loss of outward radial force available to do useful work.

By placing one standard shape balloon component inside another and by including an inner support member positioned radially inward of both balloons a hollow balloon component that allows for perfusion while the balloons are inflated is realised. Such an invention is described in US20200179116.

### SUMMARY OF INVENTION

This invention provides a medical device, such as, for example, a balloon catheter, an introducer sheath or an implant, which includes a hollow balloon component and which can be actuated between an inflated state and an un-inflated or contracted state by means of an inflation lumen contained within a catheter to which the balloon module is affixed.

More specifically, the invention provides a novel configuration of balloon component which has a body which, in one embodiment, has at least one conical section and, in another embodiment, has a conical section and a cylindrical working section, and a cavity spaced radially inwardly of the conical section or conical section and cylindrical section.

In the inflated state, the body of the balloon module may take on an overall shape that is largely similar to either a standard shape balloon component (as described in the background section), having a first neck region which transitions into a first cone region, which transitions into a first working region, which transitions into a second cone region, which transitions into a second neck region; or a funnel shaped balloon component (as described in the prior art) having a first neck region which transitions into a first cone region, which optionally transitions into a working region.

The balloon module is configured to be mounted on or affixed to or bonded to a catheter.

The catheter may include an inflation lumen and/or a working passage or working channel.

The body of the balloon component may be comprised of an outer layer or wall and an inner layer or wall, between which an inflatable enclosure is defined and which enclosure is in fluid communication with the inflation lumen of the catheter to which the balloon module is affixed.

The inflatable enclosure may be inflated by ingress of an inflation medium passed along the inflation lumen to move from the un-inflated state to the inflated state.

Unlike the prior art, at least one portion of the inner wall may be bonded directly, by any suitable method such as, for example, a thermal-bond or thermal-weld, to at least one portion of the outer wall to provide at least one bond zone.

It is an objective of the invention that, upon inflation of the inflatable enclosure between the inner wall and the outer wall, the at least one bond zone prevents or limits the radially inward expansion of the inner wall to provide a cavity positioned radially inward of the inner wall that beneficially remains open in the inflated state and is not impinged upon by ingress of an expanding or collapsing inner wall.

From a first perspective, the invention provides a medical device which includes a balloon module which has:
a first neck section which comprises an outer neck wall and an inner neck wall, each adapted to be engaged to a catheter shaft to enclose an outlet to an inflation passage of the catheter shaft;
a first cone section which comprises an outer cone wall, as an extension of the outer neck wall, and an inner cone wall, which is an extension of the inner neck wall;
a closed perimeter about which the outer cone wall and the inner cone wall are seal-ably connected; and
an inflation enclosure between the outer cone wall and the inner cone wall; and
wherein at least one portion of the inner cone wall is bonded directly, by any suitable method, to at least one portion of the outer cone wall to provide at least one bond zone.

The inner cone wall may be connected to the outer cone wall along the closed perimeter such that the cavity is completely defined within the cone region and is positioned radially inward from the inner cone wall.

Alternatively, the balloon module may include a working section which comprises an outer working wall, as an extension of the outer cone wall, and an inner working wall, as an extension of the inner cone wall.

The inflation enclosure in this alternative may be between the outer cone wall, the inner cone wall, the outer working wall and the inner working wall.

The working section region may take on an outer shape that is largely cylindrical, spherical or barrel-shaped. The catheter may include a working passage which may terminate near the transition of the inner neck wall to the inner cone wall. Alternatively, the working passage may extend distally beyond the neck region. Alternatively, the working passage may extend beyond the working region.

The outer working wall may be connected to the inner working wall continuously about the closed perimeter.

The working passage may terminate near the transition of the inner neck wall to the inner cone wall.

Alternatively, the balloon module may include a working section which comprises an outer working wall, as an extension of the outer cone wall, and an inner working wall, as an extension of the inner cone wall.

Further alternatively, the balloon module may include a second cone section, extending from the working section, and terminating at a second neck section. With the outer working wall connected to the inner working wall continuously about the closed perimeter, second cone section and the second neck section are not inflatable and may comprise of a single wall.

Preferably, the working passage terminates distally of the second neck section.

The first cone section may include at least aperture which is configured to allow for fluid flow or perfusion there-through or for other devices such as, for example, guide-wires, guide catheters or delivery-catheters to pass there-through when the balloon module is inflated.

The at least one aperture may be sealed about its respective perimeter with the bonding of the outer cone wall to the inner cone wall.

The balloon module may include a flexible mesh or filter or fabric which covers or extends across the at least one aperture, adapted to allow fluid, such as blood, to flow there-through whilst preventing larger particles, such as for example calcium debris from a TAVI procedure, from passing through this flexible filter.

Alternatively or additionally, the working section may include at least one aperture as described above.

Additionally, the second cone region may include at least one window cut-outs which is adapted to allow for fluid flow or perfusion there-through or for other devices such as for example guide-wires, guide catheters or delivery-catheters to pass there-through when the device is deployed and inflated.

The at least one window cut-out may be spanned by a flexible mesh or filter or fabric configured to allow fluid, such as for example blood, to flow there-through whilst preventing larger particles, such as for example calcium debris from a TAVI procedure, from passing through this flexible filter.

In one alternative, the un-inflatable second cone section may be made completely of a flexible mesh or filter adapted as described above.

The balloon module may be mounted to the catheter with the first cone region positioned distal of the first neck region.

Alternatively, the balloon module may be mounted to the catheter with the first cone region positioned proximal of the first neck region i.e. a balloon module mounted in reverse.

From a second perspective, the invention provides a medical device for use within the human body, which includes:
a catheter tube having a distal end and a proximal end;
an inflation passage defined within the catheter tube having an outlet;
at least one balloon module which has a first end and a second end which engage the catheter tube to envelop the outlet, and between which an outer wall, a closed perimeter, and an inner wall is disposed to define an inflation enclosure, and
a cavity defined within the inner wall;
wherein the balloon component is inflatable by inflow of an inflation fluid through the inlet into the inflation enclosure from a deflated state to an inflated state;
wherein the balloon has at least one bond zone within which the inner wall is bonded directly to the outer wall, and
wherein, in the inflated state, the outer wall and at least part of the inner wall move radially outwardly relatively to the longitudinal axis of the catheter to cause expansion of the cavity and to radially space apart the closed perimeter from the longitudinal axis of the catheter.

The at least one bond zone may cause the inner wall to move radially outwardly with the outer wall on inflation to expand the cavity.

The balloon component may extend in a catheter deployment or insertion direction (hereinafter, a distal direction). In this alternative, the balloon component engages the catheter tube with the first end positioned proximally relatively to the second end (hereinafter referred to as “a forward extending balloon”).

The balloon component may extend in a catheter retraction direction (hereinafter, a proximal direction). In this alternative, the balloon component engages the catheter tube with the first end positioned distally relatively to the second end (hereinafter referred to as “a backward extending balloon”).

The closed perimeter may be an edge or region of curvature or inflection defining a boundary or transition between the outer wall and the inner wall (hereinafter referred to as the “edge”). The edge may describe in a generally circumferential path. Alternatively. The edge may be jagged, wavi-linear, sinusoidal or crown-shaped or it may have a mitred or wedge-like shape (similar in shape to the sharp tip of a hypodermic needle for example)

The outlet may be located at any point along the catheter tube. Preferably, the outlet is disposed at or towards the distal end.

The catheter tube may have at least one working passage or working channel.

A guide-wire may be passed along or within the at least one working passage.

The catheter tube may include a conduit, within which the at least one working passage is defined.

The first end of the balloon component may engage the catheter tube with the second end engaging the conduit.

Alternatively, the first end of the balloon component may engage the conduit with the second end engaging the catheter tube.

The conduit may have an inlet disposed at or towards the distal end.

In an application which requires improved guidance or tracking along the vessel in which the catheter is deployed, the conduit may extend beyond the edge of the balloon component as an extending section. The conduit may include an a traumatic tip and radiopaque marker bands.

The balloon module may include a plurality of tethers, each of which extend between the edge and the extending section.

The tethers may be equally circumferentially spaced relatively to one another.

The plurality of tethers may extend between the edge and the extending section in an oblique direction. The plurality of tethers may converge at a tubular neck adapted to be bonded an outer surface of the extending section.

The medical device may include a flexible conical filter which extends between from the edge and the extending section and which is seal-ably connected to the edge or working portion and the extending section. The flexible filter or fabric or mesh is configured to allow fluid, such as for example blood, to flow there-through whilst preventing larger particles, such as for example calcium debris from a TAVI procedure, from passing through this flexible filter.

The balloon component may be configured with a conical portion between the first and second ends and the edge.

The at least one bond zone may be a circumferentially continuous band.

The at least one circumferentially continuous band may be adjacent the edge.

The at least one bond zones may be formed by direct sealing engagement of the inner wall to the outer wall by employing any suitable method.

Alternatively, the balloon component may have a plurality of bond zones.

Each bond zone may be elongate or circular. The elongate bond zones may be longitudinally, circumferentially, helical or spirally configured. The elongate bond zones may be straight or take on a V-, W- or chevron shape. A pair of elongate bond zones may intersect or join to provide an X-shaped bond zone, or a zig-zag bond zone or an S shape bond zone or a sinusoidal bond zone.

It is anticipated within the scope of the invention that the bond zones may take on a shape which includes a combination of any one or more of the abovementioned shapes.

The balloon component may include a plurality of inflatable pockets defined between the bond zones.

At least one of the inflatable pockets may be a circumferentially continuous inflatable pocket adjacent the edge.

As an alternative to the balloon component being configured with a conical portion alone, the balloon component may be configured to include the conical portion and a cylindrical portion between the conical portion and the edge.

The plurality of bond zones may be on the cylindrical portion (or working region or working length), the conical portion or on both the cylindrical portion and the conical portion.

In a non-occlusive or perfusion embodiment, at least one bond zone may include an aperture which opens the hollow space or cavity to an exterior of the outer wall to allow the passage of fluid, like for example blood, or other devices, like for example a guidewire or guide-catheter, through the conical portion or the cylindrical or working portion of the balloon.

In the non-occlusive embodiment, one or more of the outer wall and the inner wall may be coated with a drug-containing layer.

Preferably, a plurality of bond zones may include an aperture. The plurality of apertures may be through bond zones on the conical portion, the cylindrical portion or the conical portion and the cylindrical portion.

One or more apertures may be seal-ably covered over with a flexible filter or fabric mesh configured to allow fluid, such as for example blood, to flow there-through whilst preventing larger particles, such as for example calcium debris from a TAVI procedure, from passing through this flexible filter.

The outer wall and the inner wall may be made of a first material and a second material respectively.

The first material may be a softer, more elastic material, than the second material to assist in the formation of a seal against a vessel in which the catheter is deployed, whilst maintaining the shape of the cavity.

Alternatively or additionally, the second material may be harder than the first material to limit inward expansion of the inner wall.

Further alternatively, the first material may have a higher friction coefficient or a lower friction coefficient than the second material.

If the first material has a higher friction coefficient, with the second material being more lubricious, it may enable the catheter to anchor within the vessel whilst easing movement of a body or device within the lumen.

The invention extends to a balloon capsule catheter which includes
a catheter tube having a distal end and a proximal end;
an inflation passage defined within the catheter tube; and
a balloon capsule on the catheter tube which includes a forward extending balloon and a backward extending balloon as respectively described above;
wherein the forward extending balloon and the backward extending balloon engaged with each other along respective their respective edge.

A generally distal portion of the catheter, preferably the distal portion of the outer tube, may be steerable by means of pull wires and a handle mounted at or near the hub or other methods commonly used for steerable catheters

The catheter may include an outer sheath which is adapted to slide over the balloon module when the balloon is in the deflated state. This could aid in inserting the balloon into the body or through narrow vasculature en route to the target treatment site.

### BRIEF DESCRIPTION OF THE DRAWINGS

The invention is further described by way of an examples, with reference to the accompanying drawings in which;

FIG. 1A to 1F diagrammatically illustrate various embodiments of a medical device comprising one or more balloon modules;

FIG. 2 is a view in elevation of a medical device in accordance with a first embodiment, showing a single balloon module in a conical or funnel-shaped configuration;

FIG. 3 is the medical device of FIG. 2 shown isometrically and in longitudinal section;

FIG. 3A diagrammatically illustrates the bond areas or necks of the balloon module of the medical device of FIG. 1;

FIG. 4 is an isometric view of a medical device in accordance with a second embodiment of the invention in longitudinal section, showing a single balloon module in a conical configuration;

FIG. 5 is a view in elevation of the medical device of FIG. 4;

FIG. 6 is an isometric view of a medical device in accordance with a third embodiment of the invention in longitudinal section, showing a single balloon module with a conical portion and a cylindrical working length portion;

FIG. 7 is a view in elevation of the medical device of FIG. 6, longitudinally sectioned;

FIG. 8 is an isometric view from one end of a medical device in accordance with a fourth embodiment of the invention, showing a single balloon module in a conical and a cylindrical portion;

FIG. 9 is an isometric view from the other end of the medical device of FIG. 8;

FIGS. 10 and 11 each isometrically illustrate a fifth and a sixth single-balloon embodiment of a medical device respectively;

FIG. 12 is an isometric view of a medical device in accordance with a seventh single-balloon embodiment of the invention;

FIG. 13 is an isometric view of a medical device in accordance with an eighth single-balloon embodiment of the invention;

FIG. 14 is an isometric view of a medical device in accordance with a ninth single-balloon embodiment of the invention;

FIG. 14A is an isometric view of a medical device in accordance with a tenth single-balloon embodiment of the invention which embodiment has a plurality of distal end tethers;

FIG. 15 is an isometric view of a medical device in accordance with an eleventh single-balloon embodiment of the invention which embodiment has a plurality of distal end tethers;

FIG. 16 is an isometric view of a medical device in accordance with an eleventh embodiment of the invention which embodiment which embodiment has comprises a pair of balloon modules;

FIG. 17 is a diagrammatic illustration of a medical device in longitudinal section and in accordance with a twelfth single-balloon backward extending embodiment; and

FIG. 18 is a diagrammatic illustration of a medical device in longitudinal section and accordance with a thirteenth single-balloon embodiment.

### DESCRIPTION OF PREFERRED EMBODIMENTS

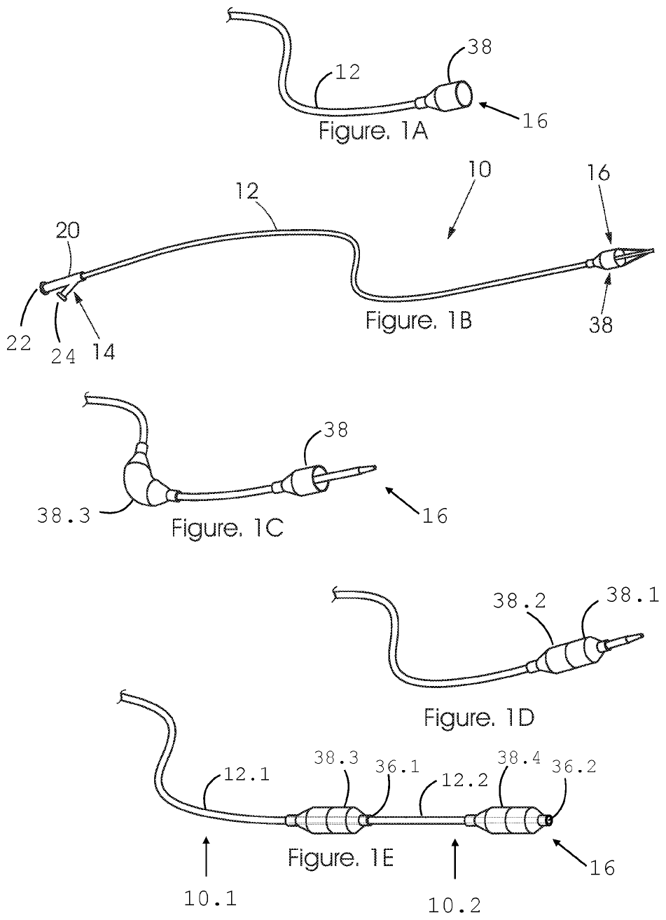

FIGS. 1A to 1 E illustrate various embodiments and arrangements of a medical device 10 in accordance with the invention.

The medical device 10 includes an elongate, flexible, catheter tube 12 which extends between a proximal end 14 and a distal end 16. The tube typically is made from extruded polymer tubing such as PEBAX, Polyurethane, Nylon 12 or a multiplayer or braid-reinforced extrusion and typically has a length of 100 mm to 1200 mm. On the proximal end, the catheter carries at the very least a Y connector or hub 20 (including one or more Luer fittings). The hub 20 includes at least two ports, a working or guidewire port 22 and an inflation port 24. The hub may house or comprise additional features or components such as a haemostasis-valve (as is common place in introducer sheaths) and a de-airing port which would allow other devise to be inserted into the working passage or retrieved via the working passage with minimal leakage of blood or other bodily fluids and whilst maintaining air ingress. The hub may also comprise an actuating mechanism for the advancement and retraction of a sheath configured to envelop the balloon module when deflated. The hub may also include an actuating mechanism for a steerable catheter, which may allow the distal end of the catheter tube 12 to be actuated from a straight state to a somewhat curled up or bent state, to aid in placement of the balloon module within the human body.

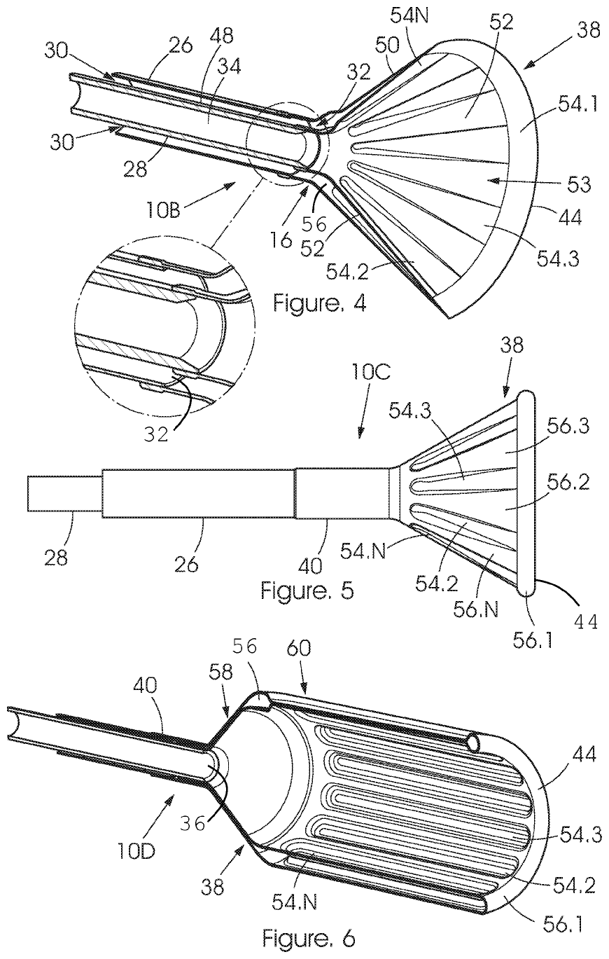

The catheter tube comprises an outer tube 26 and a generally concentric inner tube 28. An inflation passage 30 (see FIGS. 3 and 4) is defined by an annular space between the outer and the inner tube. At or near the distal end, the inflation passage has an outlet 32. The inflation passage fluidly communicates between the inflation port 24 and the outlet 32.

It should be noted what while a co-axial inflation configuration is shown in the Figures, another way to inflate the balloon module is using a multi-lumen extrusion, as is common practice and well-known to those familiar with the art of balloon catheter design and manufacture. Therefore the co-axial configuration is not intended to be limiting on the invention, nor is the distal end location of the outlet.

Within the inner tube 28, a working passage is defined 34. This passage communicates the working port 22, on the proximal end 14, with a working passage opening 36. The working passage 34 can be used as a guidewire-lumen. The working passage opening 36 can be configured to receive the proximal end of a guidewire and the working passage can be configured such that said guidewire can be fed proximally until it extends beyond the proximal end 14 and beyond the proximal end of the guidewire port 22. The working passage opening 36 can be configured to allow translation of a second (smaller version) of a balloon module of the invention there through, such that the second balloon module can be slidably and rotationally actuated with respect to a first balloon module.

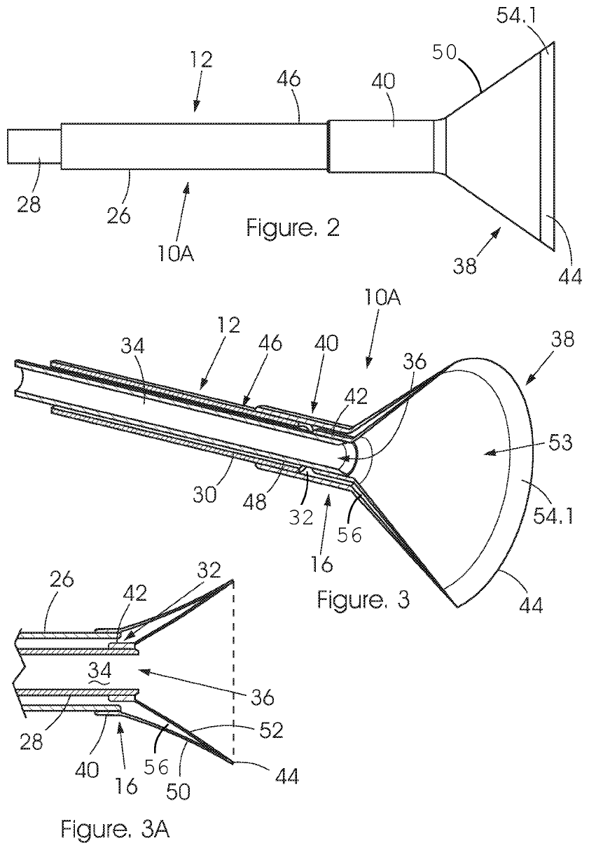

As illustrated in FIGS. 1A, 1B and FIG. 2, on the distal end of the catheter tube 12, the device 10 has a single balloon module 38. However, it is contemplated within the scope of the invention that the balloon module can be located on catheter tube 12 at any point between the proximal and distal ends (14, 16). In this respect, see FIG. 1C. Furthermore, the balloon module can be mounted to the catheter shaft in a forward facing or in a reverse facing direction.

To illustrate that the balloon module's position on the catheter tube, the orientation of the balloon module, the shape of the balloon module and the number of balloon modules are non-limiting on the invention, see FIGS. 1A to 1E. In FIG. 1D, two balloon modules are illustrated, a single balloon module 38.1 and a composite balloon module 38.2. In FIG. 1C, a composite balloon module 38.3, comprising two single balloon modules is illustrated. In FIG. 1D, the single balloon module 38.1 is a backward facing module. A backward facing balloon module 38.1 is positioned distal of a forward facing balloon module 38.2 the single balloon module 38.1 is a backward facing module. In FIG. 1E a first device 10.1 including a first catheter tube 12.1 and first composite balloon module 38.3, which first device 10.1 terminates distally at a first passage opening 36.1. Protruding further distally from the first passage opening 36.1 is a second device 10.2 including a second catheter tube 12.2 (configured to move slidably in a proximal or distal direction and rotationally within the first catheter tube 12.1) and a second composite balloon module 38.4 and a second passage opening 36.2. Both the first device 10.1 and the second device 10.2 could include within their respective catheter tubes, a steerable catheter using for example pull wires, to actuate each catheter tube and allow for navigation of the device through tortuous anatomy as is customary in the field of percutaneous guide catheters and delivery systems.

As best illustrated in FIGS. 3, 3A and 18, the balloon module has a first and second end (respectively designated 40 and 42), an closed perimeter 44 (which in some embodiments is an inflection edge, in others it is a curved or rounded rim), an outer wall 50 between the first end and the perimeter and an inner wall 52 between the perimeter and the second end. A lumen, hollow region or cavity 53 is defined within the inner wall.

The balloon module 38 is sealable affixed to the catheter at both the first end 40 and the second end 42 to envelop the outlet 32 to the inflation passage 30. In the examples, the first end is circumferentially affixed in a band (hereinafter referred to as “the neck”) to an outer surface 46 of the outer tube 26, and the second end is circumferentially affixed in a band to the outer surface 48 of the inner tube 28. In this manner, the annular-shaped outlet is enveloped. The balloon module transitions from a conical shape to a tubular shape at both zones of affixation towards the proximal end 14 as illustrated in FIG. 3A. In FIG. 18, however, with the inner tube extending significantly beyond the outer tube, the zone of affixation about the second end extends towards the distal end 16.

Configuring the balloon module as shown in FIG. 18 may allow for easier manufacture as it would avoid the need to invert the second end 42 or to evert the first end 40 prior to assembly onto the catheter, a task that can pose significant challenges especially if the balloon module is made from a relatively hard material like PET or Nylon 12 for example.

By introducing an inflation medium or fluid, such as saline (or saline plus a contrast-additives like barium sulphate can be mixed with the saline to allow for visualisation of the balloon module under X-Ray or Fluoroscopy) water, air, nitrogen or carbon dioxide into the inflation port 24, which flows along the inflation passage 30 and exits the outlet 32 into an interior (an inflation enclosure 55) of the balloon module, the balloon module can be inflated from a deflated state to an inflated state. The deflated state is significantly less voluminous (or smaller in diameter) than the inflated state as is the case with typical medical balloons. The balloon module can thus be actuated into the deflated state in which it is substantially smaller in diameter and displaces a lesser volume than when in the inflated state. This functionality is similar to a standard balloon component and has the same benefits in that the medical device 10 can be inserted into the human body (usually via an introducer sheath or port) and maneuvered into the target location within the human body while in the deflated state. Once the balloon module is in the target location, it can be inflated to perform useful work.

A characterising feature of the medical device 10 of the invention, however, is that the outer wall 50 of the balloon module 38 is bonded to the inner wall 52 within at least one bond zone 54. By way of initial example, in the first embodiment, illustrated in FIGS. 2 and 3, in which the balloon takes on a conical shape, the at least one bond zone is a circumferentially continuous band 54.1 adjacent the closed perimeter 44.

The balloon module 38 can be made from a polymer such as, for example, polyurethane, nylon-12, PEBAX, PET (Polyethylene terephthalate) or THV (Polymer comprising tetrafluoroethylene, hexafluoropropylene & vinylidene fluoride for example). The polymer can also include fibre reinforcement for improved resistance to picture or burst at higher inflation pressures. The balloon module can be made using compliant or semi-complaint medical balloon materials such as Polyurethane, Pebax or a Thermoplastic Elastomer (TPE); or a non-compliant medical balloon materials such as PET (Polyethylene terephthalate) or Nylon 12. The balloon module also can be made using one material for the outer wall 50 and another material for the inner wall 52.

The balloon module 38 can be manufactured using balloon components or elements made using a blow-forming process or using a dip-casting or dip-moulding process or using an injection moulding process. The bonding of the outer wall 50 to the inner wall 52 can be achieved using heat-bonding, thermal bonding, ultrasonic-welding, an adhesive, rivets, staples, clips, hoops, woven stitches or thread or any combination of the aforementioned.

On inflation, moving from the deflated state to the inflated state, the balloon will take an inflated-shape producing a measurable outward radial force, on walls of whatever body cavity or vessel the balloon catheter is deployed within, whilst maintaining the hollow region or cavity within the inner wall 52.

By affixing, joining or bonding the outer wall 50 to the inner wall 52, the inner wall is significantly prevented from elongating, crumpling, buckling or yielding inwards (preventing occlusion of the lumen, hollow region or cavity 53). Being connected in this manner, the outer wall 50 produces an outward radial force, on inflation of the balloon module, and pulls the inner wall 52 radially outwardly with it. When the balloon is inflated, the net outward radial force is roughly equal to the inflation pressure multiplied by the area of the outer wall (“outward facing area”) minus the area of the inner wall (“inward facing area”). Since the balloon component will usually be configured to provide an outward area larger than the inward area, the net force produced by this invention when inflated with positive pressure, is an outward radial force This outward radial force maybe useful for creating a seal against the inner diameter of a vessel, and/or opening up or dilating a vessel or a stented-implant such as, for example, a stent or a balloon expandable TAVI valve or a stent-graft for treating aneurysms through a percutaneous minimally invasive procedure.

The medical device 10 of the invention can be used in many medical treatment applications such as, for example; as a retrieval device to filter-out or capture or retrieve or remove calcium deposits or fragments from the human body similar to a cerebral protection system, to retrieve other medical devices such as, for example, balloon catheters or as a dilation perfusion balloon to provide an outward radial force onto a vessel (like employing a valvuloplasty procedure) or onto an implant (like employing a balloon expandable TAVI procedure) within the human body whilst maintaining perfusion when in the inflated state, or as a drug delivery device with the outer wall of the balloon module 38 coated in a drug and, on inflation of the balloon module, this wall makes contact with a target anatomy like a blood vessel for example to deliver the drug to the target anatomy, whilst maintaining perfusion while inflated.

The working passage 34 can be used to introduce a flushing fluid or pressurising fluid or a contrasting fluid or as a working channel or conduit through which other devices can pass through like, for example, a guidewire or an Endovascular Snare System (designed to retrieve and manipulate foreign objects within the body cavity) or another balloon catheter to name a few.

Reverting to the first embodiment 10A, illustrated in FIGS. 2 and 3, here the outer wall 50 is continuously seal-ably connected to the inner wall 52, in a continuous circumferential band 54.1, with an inflatable region 56 located behind this band. The balloon of this embodiment can be manufactured by inverting or everting one neck of a conical shape medical balloon (as described in the background) to form the first end 40 or the second end 42 of the balloon module.

Providing a balloon module with a circumferential band 54.1 has a benefit in that no portion of a balloon component used to make the balloon module needs to be inverted or everted to provide the outer and the inner walls. Both walls can be blow-formed or made individually as a balloon component, and then cut circumferentially on or near the working region and assembled together in the orientation illustrated in FIGS. 2, 3, 4, 9, 11, 12, 14, 15, 15, 16 and 17, and then the outer and inner walls (50 & 52 respectively) can be connected or bonded together along the band 54.1

It is beneficial to manufacture the balloon module 38 in this way because it is not easy, and sometime not possible, to invert or evert the balloon when the balloon is made of harder materials like Pebax, Nylon 12 and PET. Furthermore this manufacturing method allows for a different material to be used for the outer vs inner walls, having advantages described below.

Having the balloon 38 made in this manner allows for the introduction of harder and stronger materials which is beneficial for higher pressure balloon applications for example when a significant outward radial force is needed to open a constricted vessel, deploy a stent, post-dilate a stent or deploy or post dilate a stented-heart-valve. Furthermore, this manufacturing method makes it possible to form each wall (50, 52) from a distinct material, for example the outer membrane could be made from a Pebax 72D material, while the inner membrane could be made from a softer more elastic Pebax 63D material. The benefit here is that each surface can then be optimised to promote improved functionality. For example, the inner wall 51 can be made from a lubricious material like THV, such that it is easier to pull foreign objects (like prior deployed medical devices) into it, while the outer wall 50 can be made from a higher friction coefficient material like Polyurethane, to promote anchorage within the vessel.

Alternatively, the inner wall 52 can be made from a stronger and harder material (like Nylon 12 or Pebax 72D), less likely to collapse inwards under the inflation pressure, while the outer wall 50 can be made from a softer more elastic material (like Polyurethane with shore hardness 70A-80A or Pebax 35D) which is better suited to expand outwards under pressure like a standard compliant balloon component. This would allow for the outer wall to elongate and form a more comprehensive seal with the target vessel or device or implant within which it is inflated, without compromising the inner hollow shape of the lumen 53.

In a second embodiment 10B, illustrated in FIG. 4, the balloon has a plurality of bond zones. It has the circumferential band 54.1, with the concomitant benefits described above, and a series of substantially longitudinally aligned elongate bond lines (54.2, 54.3 . . . 54N) which radiate from the passage opening 36 to the band 54.1.

This pattern is not limiting on the invention and equally, the bond zones can be oriented to be circumferential, helical, spiral, with each zone either straight, taking on a V-, W- X- or chevron shape, an S-shape, a zig-zag shape or a circular shape or any combination of the foregoing.

In a third embodiment 10C, illustrated in FIG. 5, is similar to the preceding embodiment but differs in not having the circumferential band seal 54.1. Here, the closed perimeter 44 region is un-bonded, creating an inflatable pocket 56.1 which, in this embodiment, is fully circumferentially continuous and which feature promotes sealing with a vessel in which the catheter 10C is deployed or exerting an outward radial dilating force on the vessel or on an implant.

In addition to the inflatable pocket 56.1, between the longitudinally aligned bond zones (54), there is a plurality of inflatable pockets (respectively designated 56.2, 56.3, 56.N) which allow fluid communication between the outlet 312 and the inflatable pocket 56.1.

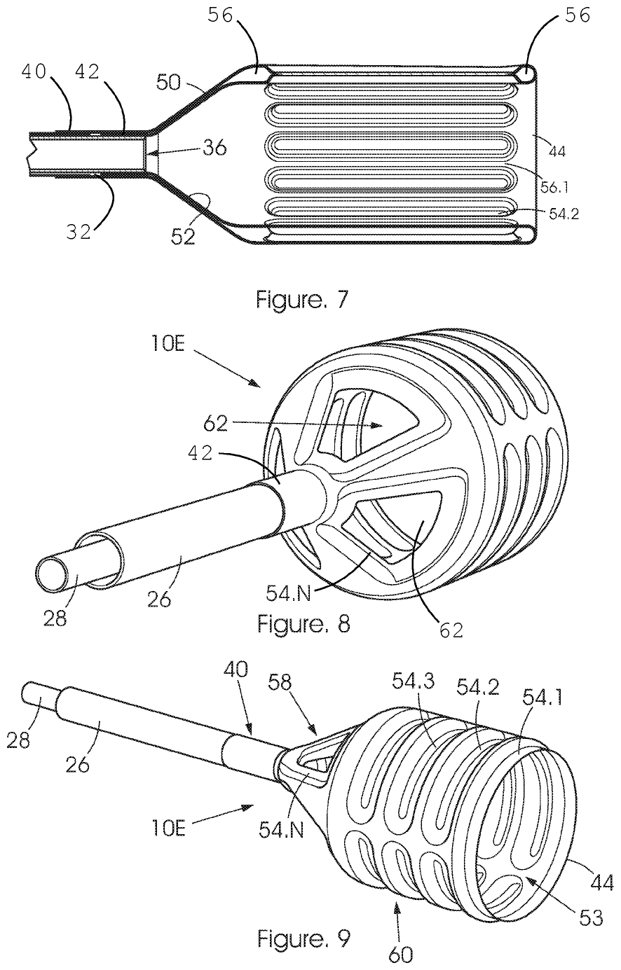

A fourth embodiment 10D is illustrated in FIGS. 6 and 7. Unlike earlier described embodiments, this embodiment is not cone shaped. Emerging from the neck 40, the balloon has a first conical portion 58 and a cylindrical portion 60, which portion defines a working length or working region. The cylindrical portion terminates at the closed perimeter 44. This embodiment has been illustrated with the cylindrical portion having a length that is similar to the diameter of same cylindrical portion. It could however be advantageous for the length of the cylindrical portion to be significantly greater than the diameter of the cylindrical portion. If we designate the working length (length of the cylindrical portion) to have a value “L” and the inflated outer diameter of the cylindrical portion to have a value “D” (the outer diameter of the inflated balloon module). A long balloon module could be configured to have D<L<300 D. This could be advantageous for retrieving long devices such as long balloon catheter for example.

In this embodiment, the balloon is configured with the circumferentially continuous inflatable pocket 56.1 and a plurality of longitudinally aligned bond zones (54.2 to 54.N) formed on the cylindrical portion 60.

FIGS. 8 and 9 illustrate a fifth embodiment 10E of the invention. Here the bond zones 54.N divergently run up along and arc around the conical portion 58 of the balloon and the bond zones 54.1, 54.2, 54.3 and 54.4 extend circumferentially but discontinuously around the cylindrical portion 60. Being discontinuous, that the pockets 56 are interconnected or in fluid communication and can therefore be pressurised. Being circumferential such that the pockets 56 form an inflatable arch-like structure which provides a structurally superior configuration capable of exerting larger outward radial forces, suitable for dilation applications.

Within the bond zones on the conical portion, cut-outs or apertures 62 are formed. These window cut-outs serve two key functions: they allow the balloon to be inflated, without occluding the vessel within which it has been inflated (this could be used to dilate a sensed vessel or valve (valvuloplasty) or to deploy a stent, stented-heart-valve, or stent-graft for treatment of aneurysm without occluding blood flow); and they remove material from the balloon module reducing the total amount of material needed, thereby improving the balloon's ability to fit into a small space when deflated which is advantageous for insertion through a guide-catheter, sheath, port or working channel of another device like an endoscope for example.

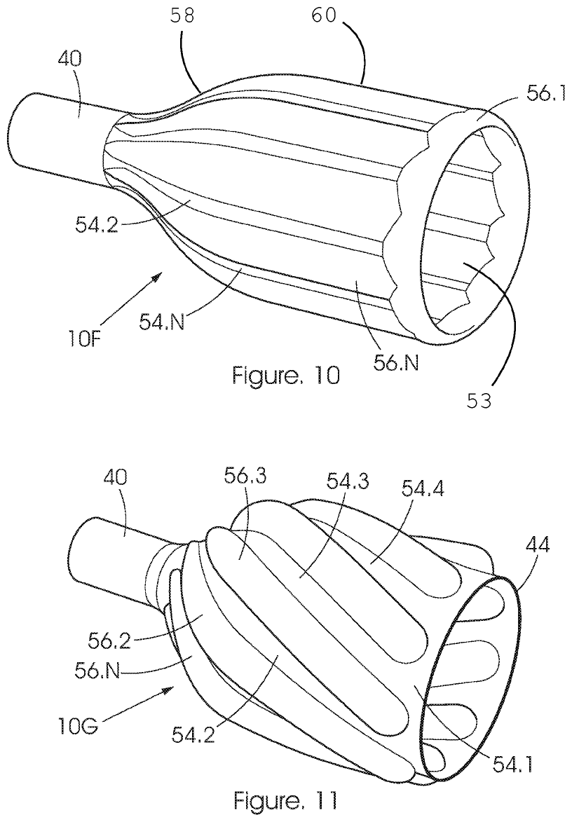

Moreover, this particular non-occlusive embodiments (see FIGS. 8, 9, 14 and 15) can be used as a drug-delivery mechanism. At least the outer wall 50 of the balloon 38 can be coated with a drug-containing layer (not shown) for the local application of a drug to a target delivery site with a vessel in which the catheter 10 is deployed. Catheters 10E, 10J and 10K, for example, having apertures, will allow the perfusion of blood and therefore allowing these embodiments to be used for drug delivery over a relatively long period of time. There is very little danger of blood flow occlusion if the fully inflated balloon module 38 of these catheters is left deployed within the vessel for anytime between 1 minute and 24 hours, depending on the indications of the particular drug. And, with the drug-containing layer on at least the outer wall, and with the outer wall coming into contact with the vessel wall, the drug can be delivered to and through the blood vessel wall in a location-specific and efficient manner, whilst maintaining perfusion. FIG. 10 illustrates embodiment 10F. Here the connected regions run longitudinally starting at or near the base of the conical region and terminating just short of the distal end of the balloon module. This results in one or more longitudinal inflatable pockets as well as a circumferential inflatable pocket at the distal end. The configuration of this embodiment allows for fluid to pass more easily into and out of the balloon module. It can also improve “de-airing” of the balloon module, a practice that is common place with balloon devices or balloon catheters which must be inflated within blood circulatory system. Adequate de-airing means that accidental failure or rupture of the balloon module should not result in air-ingress. Air pockets or bubbles can cause severe complications within the blood stream.

Instead of the circumferential inflatable pocket 56.1, this feature can be replaced with the circumferential band 54.1 depending upon application.]

FIG. 11 illustrates embodiment 10G. Here the bond zones (54.2, 54.3, and 54.4) run helically starting at or near the base of the conical region and terminating at the closed perimeter 44 of the balloon module. There is also a fully circumferential bond zone 54.1 at the distal end of the balloon module. This results in one or more helical inflatable pockets (56.1, 56.2, 56.N). The configuration of this embodiment allows for the easy passage of fluid into and out of the balloon module as is the case with embodiment 10D whilst more closely approximating the structurally superior arch-like inflatable pockets 56 and therefore providing a compromise or hybrid offering.

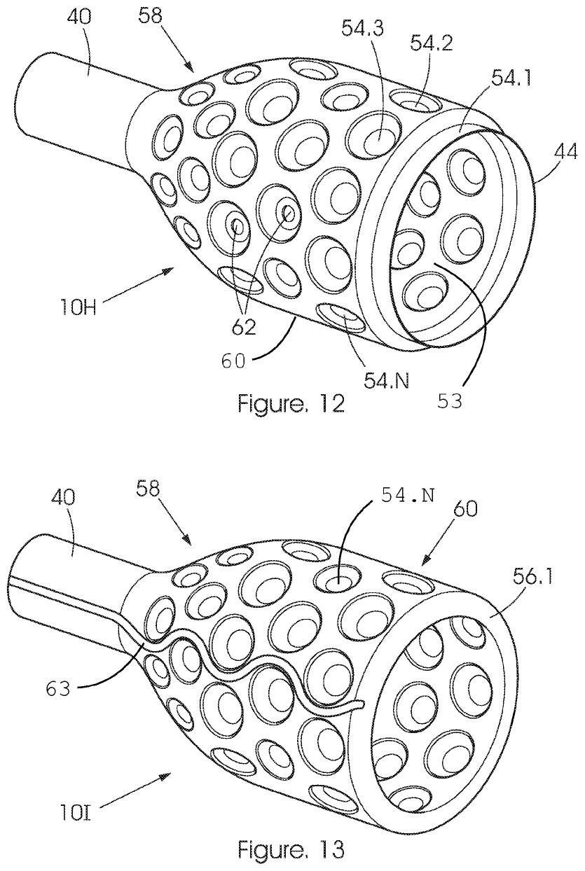

Embodiment 10H is illustrated in FIG. 12. Here the bond zones are circular in shape, starting at or near the base of the conical region or where the neck meets the cone, and terminating just short of the closed perimeter 44 of the balloon module. This balloon module has a single circumferential bond zone (band) 54.1 adjacent the closed perimeter 44. These circular shaped bond zones can include windows or cut-outs 62 to provide for perfusion. These windows can be positioned on the cone region or on the working length (cylindrical) region or on both of these regions.

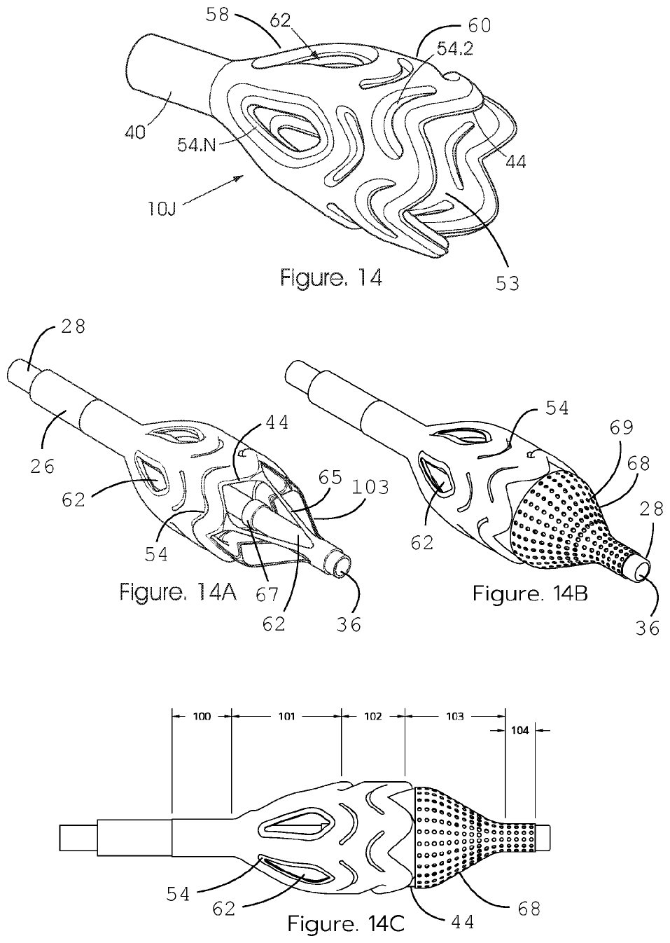

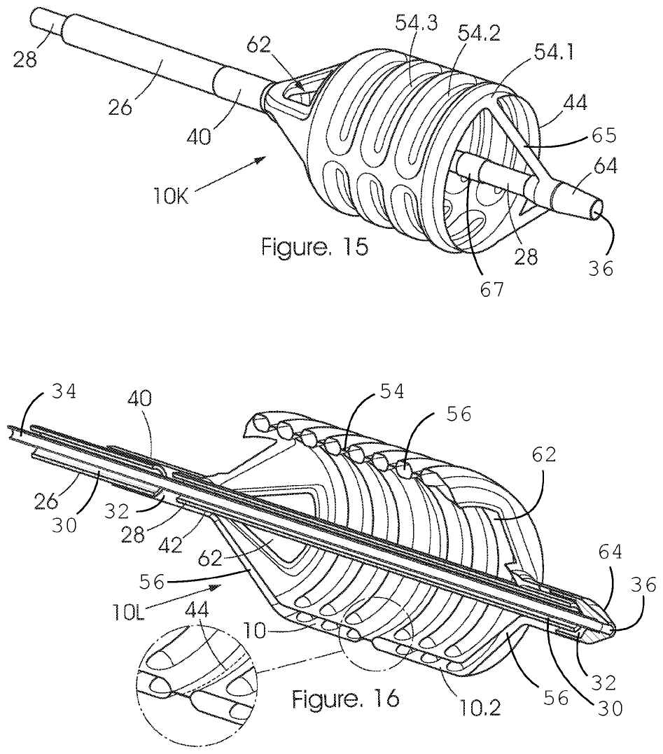

Embodiment 101 is illustrated in FIG. 13. This embodiment is similar to the preceding embodiment in that the bond zones are circular in shape, starting at or near the base of the conical region or where the neck 40 meets the cone, and terminating just short of the closed perimeter 44 which is rounded, forming part of the inflatable pocket 56.1. This embodiment includes a bond 63 which runs the full length of the balloon module. This bond can have a zig-zag shape or it can take on a helically wrapping pattern about the balloon module. The bond 62 is a feature of the manufacturing process employed in making this balloon module wherein a single sheet of polymer material first folded to create an inner and an outer layer or wall, then is rolled-up about a longitudinal axis and the edges of the sheet sealed along the bond 63 to provide the balloon module.

Another embodiment 10J is illustrated in FIG. 14. In this embodiment, the closed perimeter 44 is sealed but, unlike with earlier embodiments, the seal is not a circumferential band, but rather takes on a circumferential crown shape. The bonds on this embodiment may take on a chevron shape or an s-shape or a zig-zag shape or a partially circumferential crown shape, in order to allow the bond to flex and thereby lengthen circumferentially as the balloon module is inflated. This in turn will allow the outer diameter of the balloon module to increase as it is inflated. This feature provides for a more “compliant” (see definition in background) balloon module to be manufactured. The window cut-outs on the cone of this embodiment have a diamond shape which can be advantageous during insertion or removal of the balloon module in the deflated state. This is because the diamond shape avoids an unsupported circumferential edge, which can be difficult to pass through a narrow passage like for example an introducer sheath.

FIG. 14A shows a balloon module similar to FIG. 14, but drawn to show including an inner tube 28 which extends in both directions beyond the closed perimeter 44. This inner tube has marker bands 67 positioned on it to demarcate the working portion 102 or working length of the balloon module under X-Ray or fluoroscopy. Connected to the closed perimeter are a plurality of tethers 65, configured such that diamond shaped cut-outs or apertures 62 are included in the second cone region 103 and in part of the working region 102 also sometimes known as the cylindrical portion 60. These tethers converge at a second neck region 104 (drawn to be distal in this figure) neck which is bonded to the inner tube 28. This serves to centralise the distal end of the balloon module with respect to the inner tube 28 or a guidewire positioned therein. These tethers also serve to improve insertion of the balloon module through an introducer port. The diamond shaped cut outs along with the crown shaped closed perimeter and the tethers, avoid a large and un-supported circumferential edge, which could otherwise create difficulty during device insertion into or retrieval from the human body.

FIG. 14A shows a balloon module similar to FIG. 14, but drawn to show an inner tube 28 which extends in both directions beyond the closed perimeter 44. This inner tube has marker bands 67 positioned on it to demarcate the working portion 102 or working length of the balloon module under X-Ray or fluoroscopy. Connected to the closed perimeter are a plurality of tethers 65, configured such that diamond shaped cut-outs or apertures are included in the second cone region 103 and in part of the working region 102 (also sometimes known as the cylindrical portion 60). These tethers converge at second neck region 104 (drawn to be distal in this figure) which is bonded to the inner tube 28. This serves to centralise the distal end of the balloon module with respect to the inner tube 28 or a guidewire positioned therein. These tethers also serve to improve insertion of the balloon module through an inroducer port. The diamond shaped cut outs 62 along with the crown shaped closed perimeter and the tethers, avoid a large and un-supported circumferential edge, which could otherwise create difficulty during device insertion into or retrieval from the human body.

FIG. 14B shows a balloon module similar to FIG. 14A but including a distal cone region 103 and a distal neck region 104 which are formed from the same part 68 which is made from a flexible mesh or filter or fabric shown to have small perforations 69 (which have been scaled up for visualisation and may in reality not be visible to the naked eye). This conical filter 68 is bonded to the closed perimeter or edge of the balloon module and also to the outer surface of the inner tube 28. This conical flexible filter can be configured to allow fluid (such as for example blood) to flow there through whilst preventing larger particles (such as for example calcium debris from a TAVI procedure) from passing through this flexible filter. With this added feature, the balloon module could be positioned such that a bodily fluid like blood is free to flow into the hollow space or cavity 53 via the window cut-outs or apertures positioned on the first cone region 101. Once within the hollow space or cavity it is forced through the filter by the upstream pressure. Any particulate that is too large to pass through the filter would thus remain trapped within the hollow space or cavity within the balloon module. Once the procedure is complete the balloon module could be returned to its deflated state and retrieved, along with the filtered particulate. FIG. 14C is a side on view of the same embodiment illustrated in FIG. 14B but it is intended to describe the general regions of the invention. This embodiment includes a first neck region 100, a first cone region 101, a working region 102 and a second cone region 103 and a second neck region 104. All embodiments include regions 100 and 101 while only some embodiments include regions 102, 103, 104.

FIG. 15 shows a balloon module similar to that shown in FIGS. 8 and 9 but this embodiment 10K is configured such that the working passage opening 36 is positioned distal of the closed perimeter 44. This could allow for radio-opaque marker bands 67 to be positioned or connected or bonded to the inner tube 28. These marker bands could be aligned with the proximal and distal ends of the cylindrical portion 60 (also known as the working region 102) of the balloon module. This could allow for the balloon module to be properly located by means of X-ray or Fluoroscopy prior to inflation of the balloon module, as is customary with many balloon catheters. This embodiment could further include a plurality of tensile elements or tethers 65 (which could also take the form of a second cone with window cut-outs for perfusion as shown in FIG. 14A), both of which would serve to stabilise and centralise the distal end of the working region 102 or cylindrical region 60 with respect to the inner tube 28 and therefor with respect to a guidewire running through said inner tube. Furthermore, these tethers or second cone, could also serve to make the balloon module more robust during insertion into and retrieval from an introducer sheath as they would prevent the cylindrical portion of the balloon module from crunching up or folding back on itself.

In a further embodiment 10L, illustrated in FIG. 16, two balloon modules, of the type embodied in embodiment 10E (and respectively designated 10.1 and 10.2), have been configured to form a non-occlusive dilation balloon catheter. Specifically, the two balloon modules have been bonded or connected to one another at their respective closed perimeters 44 (see the insert to FIG. 16 which illustrates this).

This embodiment could be configured to allow both balloon modules to inflate simultaneously and from the same pressure source (as drawn) or individually in a two-step inflation protocol. This two-step inflation protocol could be achieved by running the inner tube 28 all the way to the proximal end or hub 20 of the catheter, and thereby creating a second inflation lumen or second annular inflation space. This could allow the annular lumen between the outer catheter shaft and the central catheter shaft to be pressurised independently, thereby inflating the proximal balloon module but not the distal balloon module. It can be beneficial for the balloon module to inflate from a proximal side as well as from a distal side, particularly when deploying an implant. This functionality can aid in uniform deployment of the implant, with both the proximal and distal ends of the implant expanding together, as the balloon inflated from each end simultaneously.

As with the non-inflatable tethers 65 of embodiment 10K, the distal balloon module 10.2 in this embodiment functions as an inflatable tether to the proximal balloon module 10.1 in that it connects the closed perimeter 44 to the inner tube 28 with the advantages discussed above in paragraph 145.

A guidewire 66 can pass through the inner catheter shaft, and through the tip and out of the distal end of the device. Radiopaque marker bands 67 can be added to the central catheter shaft to allow positioning of the device within the target site under x-ray imaging guidance.

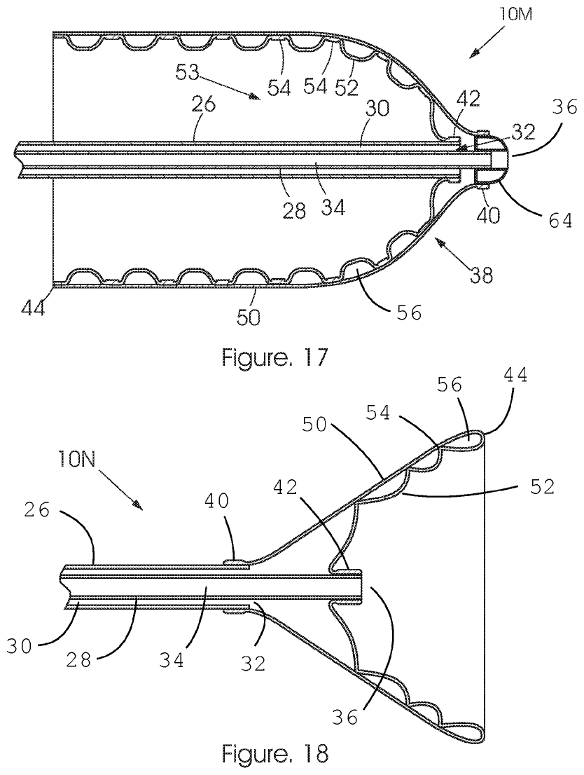

In all the preceding embodiments, all the balloons modules 38 have been illustrated to include a forward extending balloons in that the direction of extension of the balloon 38, from the ends (40, 42) to the edge or rim 44, is a catheter deployment or forward direction. However, as illustrated in FIG. 17, an embodiment 10M includes a backward extending balloon module. In this embodiment, the direction of extension of the balloon from the ends 40 and 42 to the closed perimeter 44 is in a catheter retraction direction. In this embodiment, the outer wall 50 is defined between the second end 42 and the closed perimeter 44 and the inner wall 52 is defined between the first end 40 and the closed perimeter. This embodiment is useful in circumstances where the anatomy is shaped to receive this configuration more easily than others. The embodiment also can be used to orient the balloon module in this reversed orientation to allow easier insertion of the balloon module through a narrow passage such as an introducer sheath or working channel.

Potential applications for the medical device of the invention include use in procedures such as cerebral protection, transcatheter aortic valve replacement, balloon valvuloplasty and tracheal balloon dilation among other.

1. A medical device for use within the human body, which includes a catheter tube having a distal end and a proximal end, an inflation passage defined within the catheter tube and having an outlet, at least one balloon module which has a first end and a second end which engage the catheter tube to envelop the outlet, and between which an outer wall, an inner wall, and an edge between the outer wall and the inner wall, are disposed to define an inflation enclosure, at least one bond zone within which the inner wall is bonded directly to the outer wall, and a cavity defined within the inner wall, wherein the balloon component is inflatable by inflow of an inflation fluid through the inlet into the inflation enclosure from a deflated state to an inflated state, wherein the edge is radially spaced from the longitudinal axis of the catheter such that when the balloon component moves from the deflated state to the inflated state, the outer wall and at least part of the inner wall move radially outwardly relatively to the longitudinal axis of the catheter to cause expansion of the cavity.

2. The medical device according to claim 1, wherein the edge is an edge or region of curvature or inflection defining a boundary or transition between the outer wall and the inner wall.

3. The medical device according to claim 2, wherein the edge follows a circumferential path.

4. (canceled)

5. The medical device according to claim 3 wherein the edge is mitred, chamfered or bevelled.

6. The medical device according to claim 5, wherein the catheter tube has at least one working passage.

7. The medical device according to claim 6, wherein the catheter tube includes a conduit within which the at least one working passage is defined.

8. The medical device according to claim 7, wherein the first end engages the catheter tube and the second end engages the conduit.

9. The medical device according to claim 7, wherein the first end engages the conduit and the second end engages the catheter tube.

10. The medical device according to anyone of claim 7, wherein the conduit has an inlet disposed at or towards the distal end.

11. The medical device according to anyone of claim 7, wherein the conduit extends beyond the edge of the balloon component as an extending section.

12. The medical device according to claim 11, wherein the balloon module includes a plurality of tethers, each of which extend between the edge and the extending section.

13. The medical device according to claim 12, wherein the tethers are equally circumferentially spaced relatively to one another.

14. The medical device according to claim 13, wherein the tethers extend between the edge and the extending section in an oblique direction.

15. The medical device according to claim 14, wherein the tethers converge at a tubular neck adapted to be bonded to an outer surface of the extending section.

16. The medical device according to claim 11, which includes a conical filter which extends between the edge and the extending section.

17-18. (canceled)

19. The medical device according to claim 1, which includes a plurality of bond zones.

20. The medical device according to claim 19, wherein each bond zone is elongate or circular.

21. The medical device according to claim 19, wherein each bond zone is longitudinally, circumferentially, helical, or spirally configured.

22. The medical device according to claim 20, wherein the elongate bond zones are straight or take on a V-, W- or chevron shape.

23. The medical device according to claim 20, wherein a pair of elongate bond zones intersect or join to provide an X-shaped bond zone, or a zig-zag bond zone or an S shape bond zone or a sinusoidal bond zone.

24-34. (canceled)
